(19) United States
(12) Patent Application Publication
(10) Pub. No.: US 2008/0103573 A1
(43) Pub. Date: May 1, 2008

(54) Implantable medical elongated member including wire-like fixation elements

(75) Inventor: Martin T. Gerber, Maple Grove, MN (US)

Correspondence Address: SHUMAKER & SIEFFERT, P. A., 1625 RADIO DRIVE, SUITE 300, WOODBURY, MN, 55125, US

(73) Assignee: Medtronic, Inc., Minneapolis, MN (US)

(21) Appl. No.: 11/591,282

(22) Filed: Oct. 31, 2006

(51) Int. Cl. A61N 1/05

(52) U.S. Cl. 607/116; 607/126

(57) ABSTRACT

An implantable medical elongated member, such as a lead or catheter, includes an integrated fixation mechanism that expands upon implantation of the elongated member to fix the elongated member relative to a target tissue site, such as tissue within the epidural region proximate the spine or the sacral foramen or subcutaneous tissue proximate to an occipital or other peripheral nerve. The fixation mechanism may include a plurality of wire-like elements, which may be configured in a substantial helical shape. The wire-like elements may be formed from an elastic or super-elastic material, and expand radially outward when a restraint mechanism is removed following implantation of the elongated member.

### TECHNICAL FIELD

The invention relates to medical device systems and, more particularly, to elongated members in medical device systems.

### BACKGROUND

Electrical stimulation systems may be used to deliver electrical stimulation therapy to patients to treat a variety of symptoms or conditions such as chronic pain, tremor, Parkinson's disease, multiple sclerosis, spinal cord injury, cerebral palsy, amyotrophic lateral sclerosis, dystonia, torticollis, epilepsy, pelvic floor disorders, gastroparesis, muscle stimulation (e.g., functional electrical stimulation (FES) of muscles) or obesity. An electrical stimulation system typically includes one or more implantable medical leads coupled to an electrical stimulator.

The implantable medical lead may be percutaneously or surgically implanted in a patient on a temporary or permanent basis such that at least one stimulation electrode is positioned proximate to a target stimulation site. The target stimulation site may be, for example, a nerve or other tissue site, such as a spinal cord, pelvic nerve, pudendal nerve, stomach, bladder, or within a brain or other organ of a patient, or within a muscle or muscle group of a patient. The one or more electrodes located proximate to the target stimulation site may deliver electrical stimulation therapy to the target stimulation site in the form electrical signal s.

Electrical stimulation of a sacral nerve may eliminate or reduce some pelvic floor disorders by influencing the behavior of the relevant structures, such as the bladder, sphincter and pelvic floor muscles. Pelvic floor disorders include urinary incontinence, urinary urge/frequency, urinary retention, pelvic pain, bowel dysfunction, and male and female sexual dysfunction. The organs involved in bladder, bowel, and sexual function receive much of their control via the second, third, and fourth sacral nerves, commonly referred to as S2, S3 and S4 respectively. Thus, in order to deliver electrical stimulation to at least one of the S2, S3, or S4 sacral nerves, an implantable medical lead is implanted proximate to the sacral nerve(s).

Electrical stimulation of a peripheral nerve, such as stimulation of an occipital nerve, may be used to mask a patient's feeling of pain with a tingling sensation, referred to as paresthesia. Occipital nerves, such as a lesser occipital nerve, greater occipital nerve or third occipital nerve, exit the spinal cord at the cervical region, extend upward and toward the sides of the head, and pass through muscle and fascia to the scalp. Pain caused by an occipital nerve, e.g. occipital neuralgia, may be treated by implanting a lead proximate to the occipital nerve to deliver stimulation therapy.

In many electrical stimulation applications, it is desirable for a stimulation lead to resist migration following implantation. For example, it may be desirable for the electrodes disposed at a distal end of the implantable medical lead to remain proximate to a target stimulation site in order to provide adequate and reliable stimulation of the target stimulation site. In some applications, it may also be desirable for the electrodes to remain substantially fixed in order to maintain a minimum distance between the electrode and a nerve in order to help prevent inflammation to the nerve and in some cases, unintended nerve damage. Securing the implantable medical lead at the target stimulation site may minimize lead migration.

### SUMMARY

In general, the invention is directed towards an implantable medical elongated member that includes a fixation mechanism with a plurality of wire-like elements that are expandable to fix the elongated member proximate to a target therapy delivery site, as well as a method for implanting the elongated member. At least two of the wire-like elements are axially displaced from each other (i.e., have different axial locations along the elongated member). The elongated member is configured to be coupled to a medical device to deliver a therapy from the medical device to target therapy delivery site in a patient. The therapy may be electrical stimulation, drug delivery, or both.

For example, in one embodiment, the elongated member is an implantable medical lead that is coupled to a an external or implantable electrical stimulator, which is configured to deliver electrical stimulation therapy to a target stimulation site in a patient via the lead, and more specifically, via at least one electrode disposed adjacent to a distal end of a lead body of the lead. In another embodiment, the elongated member is a catheter configured to deliver a fluid, such as pharmaceutical agents, insulin, pain relieving agents, gene therapy agents, or the like from an external or implantable fluid delivery device (e.g., a fluid reservoir and/or pump) to a target tissue site in a patient.

In one embodiment, the invention is directed toward an apparatus comprising an implantable elongated member configured to be coupled to a medical device to deliver a therapy from the medical device to a target therapy delivery site in a patient and a fixation mechanism mechanically coupled to the elongated member. The fixation mechanism comprises a first wire-like element configured to expand to engage with tissue of the patient, and a second wire-like element axially displaced from the first wire-like element along a length of the elongate member and configured to expand to engage with the tissue of the patient.

In another embodiment, the invention is directed toward an electrical stimulation system comprising an implantable electrical stimulator and a lead comprising a lead body having a proximal end and a distal end, at least one stimulation electrode located proximate to the distal end of the lead body and electrically coupled to the electrical stimulator, and a fixation mechanism mechanically coupled to the lead body. The electrical stimulator delivers electrical stimulation to a target stimulation site via the stimulation electrode. The fixation mechanism includes a first wire-like element and a second wire like element separated from the first wire-like element by at least one stimulation electrode. The first and second wire-like elements are each expandable to substantially fix the lead body at the target stimulation site.

In yet another embodiment, the invention is directed toward a method for implanting an elongated member in a patient. The elongated member comprises a fixation mechanism mechanically coupled to the elongated member, where the fixation mechanism comprises a first wire-like element configured to expand to engage with tissue to substantially fix the elongated member proximate to a target therapy delivery site and a second wire-like element configured to expand to engage with tissue to substantially fix the elongated member proximate to the target therapy delivery site. The first wire-like element is axially displaced from the second wire-like element. The method comprises inserting the elongated member into a patient and removing a restraint mechanism on the fixation mechanism, thereby permitting the wire-like elements to expand and extend from the elongated member.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1A is a schematic perspective view of a therapy system, which includes an electrical stimulator coupled to a stimulation lead, which has been implanted in a body of a patient proximate to a target stimulation site.

FIG. 1B is an illustration of the implantation of a neurostimulation lead at a location proximate to an occipital nerve.

FIG. 2 is a block diagram illustrating various components of an electrical stimulator and an implantable lead.

FIG. 3A is a perspective drawing illustrating an exemplary neurostimulation lead that may be fixated to surrounding tissue to help prevent migration of a lead following implantation.

FIG. 3B is a perspective drawing illustrating the neurostimulation lead of FIG. 3A with the fixation mechanism in an expanded state, in which wire-like elements extend from the lead body to enable the fixation mechanism to engage with surrounding tissue, thereby fixating the neurostimulation lead proximate to a target stimulation site.

FIG. 4A is a perspective drawing illustrating an alternate neurostimulation lead with an alternative fixation mechanism attached to the lead body.

FIG. 4B is a perspective drawing illustrating the neurostimulation lead of FIG. 4A with the fixation mechanism in an expanded state, which enables the fixation mechanism to engage with surrounding tissue in order to fix a position of an implanted neurostimulation lead proximate to a target stimulation site

FIG. 5A is a perspective drawing illustrating another embodiment of a neurostimulation lead, which includes a lead body, one or more stimulation electrodes, and a fixation mechanism, which includes a number of expandable wire-like elements.

FIG. 5B is a perspective drawing illustrating the neurostimulation lead of FIG. 5A with the fixation mechanism in an expanded state, which enables fixation mechanism to engage with surrounding tissue in order to fix a position of an implanted neurostimulation lead proximate to a target stimulation site

FIG. 5C is a perspective drawing illustrating a technique for limiting the effects of fibrous ingrowth near a neurostimulation lead upon explant.

FIGS. 6A-6C are perspective drawings illustrating alternate configurations of fixation mechanisms for fixing positions of leads in accordance with the invention.

FIG. 7 is a flow diagram illustrating a process for percutaneously implanting a lead including a fixation mechanism in accordance with one embodiment of the invention.

### DETAILED DESCRIPTION

The present invention relates to an implantable medical elongated member including wire-like elements that are configured to expand upon implantation of the elongated member in a patient to substantially fix a position of the elongated member. The elongated member is configured to be coupled to a medical device to deliver a therapy from the medical device to a target therapy delivery site (e.g., proximate to a peripheral nerve) in a patient. Various embodiments of the elongated member may be applicable to different therapeutic applications. For example, the elongated member may be a stimulation lead or lead extension that is used to deliver electrical stimulation to a target stimulation site and/or sense parameters (e.g., blood pressure, temperature or electrical activity) of a patient. In another embodiment, the elongated member may be a catheter that is placed to deliver a fluid, such as pharmaceutical agents, insulin, pain relieving agents, gene therapy agents, or the like from a fluid reservoir and/or pump to a target therapy delivery site in a patient. The invention is applicable to any configuration or type of implantable elongated member that is used to deliver therapy to a site in a patient. For purposes of illustration, however, the disclosure will refer to a neurostimulation lead.

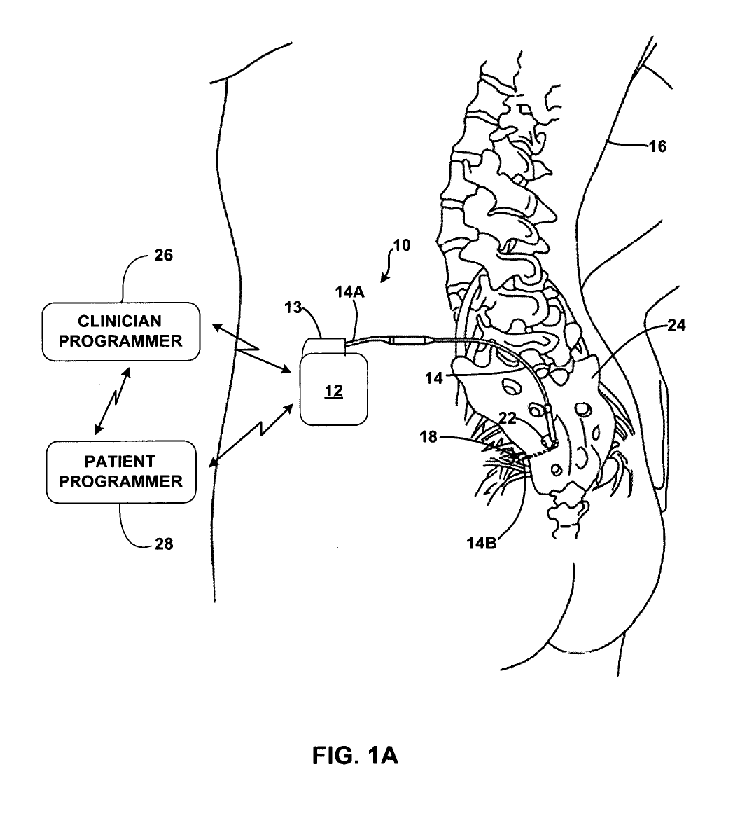

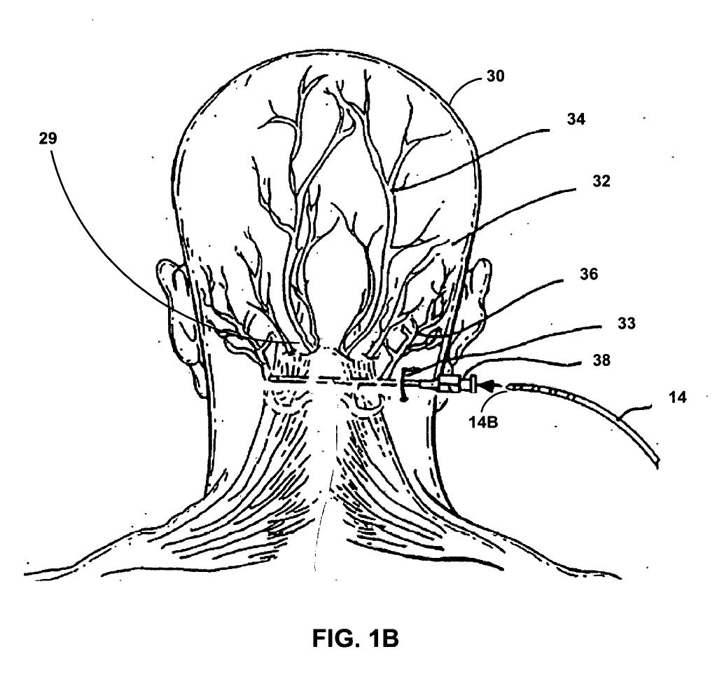

FIG. 1A is a schematic perspective view of therapy system 10, which includes an electrical stimulator 12 coupled to neurostimulation lead 14. In the example of FIG. 1A, electrical stimulator 12 implanted in body 16 of a patient proximate to target stimulation site 18. Electrical stimulator 12 provides a programmable stimulation signal in the form of electrical signals (e.g., pulses or substantially continuous-time signals) that is delivered to target stimulation site 18 by neurostimulation lead 14, and more particularly, via one or more stimulation electrodes carried by lead 14. In some embodiments, lead 14 may also carry one or more sense electrodes to permit neurostimulator 12 to sense electrical signals from target stimulation site 18. Electrical stimulator 12 may be either implantable or external. For example, electrical stimulator 12 may be subcutaneously implanted in the body of a patient 16 (e.g., in a chest cavity, lower back, lower abdomen, or buttocks of patient 16). Electrical stimulator 12 may also be referred to as a signal generator, and in the embodiment shown in FIG. 1A, electrical stimulator 12 may also be referred to as a neurostimulator. In some embodiments, neurostimulator 12 may be coupled to two or more leads, e.g., for bilateral or multi-lateral stimulation.

Proximal end 14A of lead 14 may be both electrically and mechanically coupled to connector 13 of neurostimulator 12 either directly or indirectly (e.g., via a lead extension). In particular, conductors disposed in the lead body may electrically connect stimulation electrodes (and sense electrodes, if present) adjacent to distal end 14B of lead 14 to neurostimulator 12. As described in further detail below, lead 14 further includes a lead body and at least two wire-like fixation elements (not shown in FIG. 1A) extending from the lead body, which engage with tissue to substantially fix a position of lead 14 proximate to target stimulation site 18. At least two of the wire-like fixation elements are axially displaced from each other on the lead body.

In the embodiment of therapy system 10 shown in FIG. 1A, target stimulation site 18 is proximate to the S3 sacral nerve, and lead 14 extends through the S3 sacral foramen 22 of sacrum 24 to access the S3 sacral nerve. Stimulation of the S3 sacral nerve may help treat pelvic floor disorders, urinary control disorders, fecal control disorders, interstitial cystitis, sexual dysfunction, and pelvic pain. Therapy system 10, however, is useful in other neurostimulation applications. Thus, in alternate embodiments, target stimulation site 18 may be a location proximate to any of the other sacral nerves in body 16 or any other suitable nerve in body 16, which may be selected based on, for example, a therapy program selected for a particular patient. For example, in other embodiments, therapy system 10 may be used to deliver neurostimulation therapy to a pudendal nerve, a perineal nerve, an occipital nerve (as shown in FIG. 1B) or other areas of the nervous system, in which cases, lead 14 would be implanted and substantially fixed proximate to the respective nerve.

Therapy system 10 also may include a clinician programmer 26 and a patient programmer 28. Clinician programmer 26 may be a handheld computing device that permits a clinician to program neurostimulation therapy for patient 16, e.g., using input keys and a display. For example, using clinician programmer 26, the clinician may specify neurostimulation parameters for use in delivery of neurostimulation therapy. Clinician programmer 26 supports telemetry (e.g., radio frequency telemetry) with neurostimulator 12 to download neurostimulation parameters and, optionally, upload operational or physiological data stored by neurostimulator 12. In this manner, the clinician may periodically interrogate neurostimulator 12 to evaluate efficacy and, if necessary, modify the stimulation parameters.

Like clinician programmer 26, patient programmer 28 may be a handheld computing device. Patient programmer 28 may also include a display and input keys to allow patient 16 to interact with patient programmer 28 and neurostimulator 12. In this manner, patient programmer 28 provides patient 16 with an interface for control of neurostimulation therapy by neurostimulator 12. For example, patient 16 may use patient programmer 28 to start, stop or adjust neurostimulation therapy. In particular, patient programmer 28 may permit patient 16 to adjust stimulation parameters such as duration, amplitude, pulse width and pulse rate, within an adjustment range specified by the clinician via clinician programmer 28, or select from a library of stored stimulation therapy programs.

Neurostimulator 12, clinician programmer 26, and patient programmer 28 may communicate via cables or a wireless communication, as shown in FIG. 1A. Clinician programmer 26 and patient programmer 28 may, for example, communicate via wireless communication with neurostimulator 12 using RF telemetry techniques known in the art. Clinician programmer 26 and patient programmer 28 also may communicate with each other using any of a variety of local wireless communication techniques, such as RF communication according to the 802.11 or Bluetooth specification sets, infrared communication, e.g., according to the IrDA standard, or other standard or proprietary telemetry protocols.

Therapy system 10 may also be used to provide stimulation therapy to other nerves of a patient 16. For example, as shown in FIG. 1B, lead 14 may be implanted and fixated with the two or more wire fixation members proximate to an occipital region 29 of patient 30 for stimulation of one or more occipital nerves. In particular, lead 14 may be implanted proximate to lesser occipital nerve 32, greater occipital nerve 34, and third occipital nerve 36. In FIG. 1B, lead 14 is aligned to be introduced into introducer needle 38 and implanted and anchored or fixated with fixation elements proximate to occipital region 29 of patient 30 for stimulation of one or more occipital nerves 32, 34, and/or 36. A neurostimulator (e.g., neurostimulator 12 in FIG. 1A) may deliver stimulation therapy to any one or more of occipital nerve 32, greater occipital nerve 34 or third occipital nerve 36 via electrodes disposed adjacent to distal end 14B of lead 14. In alternate embodiments, lead 14 may be positioned proximate to one or more other peripheral nerves proximate to occipital nerves 32, 34, and 36 of patient 30, such as nerves branching from occipital nerves 32, 34, and 36, as well as stimulation of any other suitable nerves throughout patient 30, such as, but not limited to, nerves within a brain, stomach or spinal cord of patient 30.

Implantation of lead 14 may involve the subcutaneous placement of lead 14 transversely across one or more occipital nerves 32, 34, and/or 36 that are causing patient 30 to experience pain. In one example method of implanting lead 14 proximate to the occipital nerve, using local anesthesia, a vertical skin incision 33 approximately two centimeters in length is made in the neck of patient 30 lateral to the midline of the spine at the level of the C1 vertebra. The length of vertical skin incision 33 may vary depending on the particular patient. At this location, patient's skin and muscle are separated by a band of connective tissue referred to as fascia. Introducer needle 38 is introduced into the subcutaneous tissue, superficial to the fascia and muscle layer but below the skin. Occipital nerves 32, 34, and 36 are located within the cervical musculature and overlying fascia, and as a result, introducer needle 38 and, eventually, lead 14, are inserted superior to occipital nerves 32, 34, and 36.

Once introducer needle 38 is fully inserted, lead 14 may be advanced through introducer needle 38 and positioned to allow stimulation of the lesser occipital nerve 32, greater occipital nerve 34, third occipital nerve 36, and/or other peripheral nerves proximate to an occipital nerve. Upon placement of lead 14, introducer needle 38 may be removed.

Accurate lead placement may affect the success of occipital nerve stimulation. If lead 14 is located too deep, i.e., anterior, in the subcutaneous tissue, patient 30 may experience muscle contractions, grabbing sensations, or burning. Such problems may additionally occur if lead 14 migrates after implantation. Furthermore, due to the location of implanted lead 14 on the back of patient's 30 neck, lead 14 may be subjected to pulling and stretching that may increase the chances of lead migration. For these reasons, fixating lead 14 may be advantageous.

In alternate applications of lead 14, target stimulation site 18 may be a location proximate to any of the other sacral nerves in patient 16 or any other suitable nerve, organ, muscle, muscle group, or other tissue site in patient 16, which may be selected based on, for example, a therapy program selected for a particular patient. For example, therapy system 10 may be used to deliver neurostimulation therapy to a pudendal nerve, a perineal nerve or other areas of the nervous system, in which cases, lead 14 would be implanted and substantially fixed proximate to the respective nerve. As further examples, lead 14 may be positioned for temporary or chronic spinal cord stimulation for the treatment of pain, for peripheral neuropathy or post-operative pain mitigation, ilioinguinal nerve stimulation, intercostal nerve stimulation, gastric stimulation for the treatment of gastric mobility disorders and obesity, muscle stimulation (e.g., functional electrical stimulation (FES) of muscles), for mitigation of other peripheral and localized pain (e.g., leg pain or back pain), or for deep brain stimulation to treat movement disorders and other neurological disorders. Accordingly, although patient 16 and target stimulation site 18 of FIG. 1A are referenced throughout the remainder of the disclosure for purposes of illustration, a neurostimulation lead 14 in accordance with the invention may be adapted for use in a variety of electrical stimulation applications, including occipital nerve stimulation, as shown in FIG. 1B with respect to patient 30.

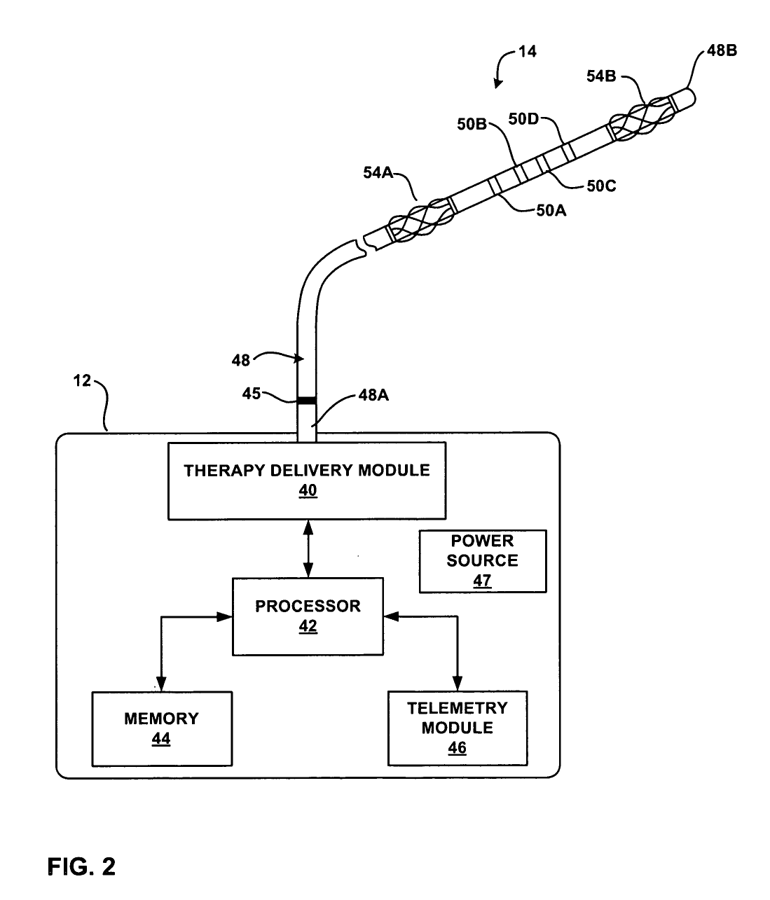

FIG. 2 is a block diagram illustrating various components of neurostimulator 12 and an implantable lead 14. Neurostimulator 12 includes therapy delivery module 40, processor 42, memory 44, telemetry module 46, and power source 47. In some embodiments, neurostimulator 12 may also include a sensing circuit (not shown in FIG. 2). Implantable lead 14 includes lead body 48 extending between proximal end 48A and distal end 48B. Lead body 48 may be a cylindrical or may be a paddle-shaped (i.e., a “paddle” lead). Electrodes 50A, 50B, 50C, and 50D (collectively “electrodes 50”) are disposed on lead body 48 adjacent to distal end 48B of lead body 48.

In some embodiments, electrodes 50 may be ring electrodes. In other embodiments, electrodes 50 may be segmented or partial ring electrodes, each of which extends along an arc less than 360 degrees (e.g., 90-120 degrees) around the circumference of lead body 48. In embodiments in which lead 14 is a paddle lead, electrodes 50 may extend along one side of lead body 48. The configuration, type, and number of electrodes 50 illustrated in FIG. 2 are merely exemplary.

Electrodes 50 extending around a portion of the circumference of lead body 48 or along one side of a paddle lead may be useful for providing an electrical stimulation field in a particular direction/targeting a particular therapy delivery site. For example, in the electrical stimulation application shown in FIG. 1B, electrodes 50 may be disposed along lead body 48 such that the electrodes face toward occipital nerves 32, 34, and/or 36, or otherwise away from the scalp of patient 30. This may be an efficient use of stimulation because electrical stimulation of the scalp may not provide any therapy to patient 30. In addition, the use of segmented or partial ring electrodes 50 may also reduce the overall power delivered to electrodes 50 by neurostimulator 12 because of the efficient delivery of stimulation to occipital nerves 32, 34, and/or 36 (or other target stimulation site) by eliminating or minimizing the delivery of stimulation to unwanted or unnecessary regions within patient 30.

In embodiments in which electrodes 50 extend around a portion of the circumference of lead body 48 or along one side of a paddle lead, lead 14 may include one or more orientation markers 45 proximate to proximal end 14A that indicate the relative location of electrodes 50. Orientation marker 45 may be a printed marking on lead body 48, an indentation in lead body 48, a radiographic marker, or another type of marker that is visible or otherwise detectable (e.g., detectable by a radiographic device) by a clinician. Orientation marker 45 may help a clinician properly orient lead 14 such that electrodes 50 face the desired direction (e.g., toward occipital nerves 32, 34, and/or 36) within patient 16. For example, orientation marker 45 may also extend around the same portion of the circumference of lead body 48 or along the side of the paddle lead as electrodes 50. In this way, orientation marker 45 faces the same direction as electrodes, thus indicating the orientation of electrodes 50 to the clinician. When the clinician implants lead 14 in patient 16, orientation marker 45 may remain visible to the clinician.

Neurostimulator 12 delivers stimulation therapy via electrodes 50 of lead 14. In particular, electrodes 50 are electrically coupled to a therapy delivery module 40 of neurostimulator 12 via conductors within lead body 48. In one embodiment, an implantable signal generator or other stimulation circuitry within therapy delivery module 40 delivers electrical signals (e.g., pulses or substantially continuous-time signals, such as sinusoidal signals) to targets stimulation site 18 (FIG. 1A) via at least some of electrodes 50 under the control of a processor 42. The implantable signal generator may be coupled to power source 47. Power source 47 may take the form of a small, rechargeable or non-rechargeable battery, or an inductive power interface that transcutaneously receives inductively coupled energy. In the case of a rechargeable battery, power source 47 similarly may include an inductive power interface for transcutaneous transfer of recharge power.

The stimulation energy generated by therapy delivery module 40 may be formulated as neurostimulation energy, e.g., for treatment of any of a variety of neurological disorders, or disorders influenced by patient neurological response. The electrical signals may be delivered from therapy delivery module 40 to electrodes 50 via a switch matrix and conductors carried by lead 14 and electrically coupled to respective electrodes 50.

Processor 42 may include a microprocessor, a controller, a DSP, an ASIC, an FPGA, discrete logic circuitry, or the like. Processor 42 controls the implantable signal generator within therapy delivery module 40 to deliver neurostimulation therapy according to selected stimulation parameters. Specifically, processor 42 controls therapy delivery module 40 to deliver electrical signals with selected amplitudes, pulse widths (if applicable), and rates specified by the programs. In addition, processor 42 may also control therapy delivery module 40 to deliver the neurostimulation signals via selected subsets of electrodes 50 with selected polarities. For example, electrodes 50 may be combined in various bipolar or multi-polar combinations to deliver stimulation energy to selected sites, such as nerve sites adjacent the spinal column, pelvic floor nerve sites, or cranial nerve sites.

Processor 42 may also control therapy delivery module 40 to deliver each signal according to a different program, thereby interleaving programs to simultaneously treat different symptoms or provide a combined therapeutic effect. For example, in addition to treatment of one symptom such as sexual dysfunction, neurostimulator 12 may be configured to deliver neurostimulation therapy to treat other symptoms such as pain or incontinence.

Memory 44 of neurostimulator 12 may include any volatile or non-volatile media, such as a RAM, ROM, NVRAM, EEPROM, flash memory, and the like. In some embodiments, memory 44 of neurostimulator 12 may store multiple sets of stimulation parameters that are available to be selected by patient 16 via patient programmer 28 (FIG. 1) or a clinician via clinician programmer 26 (FIG. 1) for delivery of neurostimulation therapy. For example, memory 44 may store stimulation parameters transmitted by clinician programmer 26 (FIG. 1). Memory 44 also stores program instructions that, when executed by processor 42, cause neurostimulator 12 to deliver neurostimulation therapy. Accordingly, computer-readable media storing instructions may be provided to cause processor 42 to provide functionality as described herein.

In particular, processor 42 controls telemetry module 46 to exchange information with an external programmer, such as clinician programmer 26 and/or patient programmer 28 (FIG. 1), by wireless telemetry. In addition, in some embodiments, telemetry module 46 supports wireless communication with one or more wireless sensors that sense physiological signals and transmit the signals to neurostimulator 12.

Migration of lead 14 following implantation may be undesirable, and may have detrimental effects on the quality of therapy delivered to a patient 16. For example, with respect to the sacral nerve stimulation application shown in FIG. 1A, migration of lead 14 may cause displacement of electrodes carried by lead 14 to a target stimulation site 18. As a result, the electrodes may not be properly positioned to deliver the therapy to target stimulation site 18, resulting in reduced electrical coupling, and possibly undermining therapeutic efficacy of the neurostimulation therapy from system 10. Substantially fixing lead 14 to surrounding tissue may help prevent lead 14 from migrating from target stimulation site 18 following implantation, which may ultimately help avoid harmful effects that may result from a migrating neurostimulation lead 14.

To that end, lead 14 further includes fixation mechanism components 54A and 54B (collectively “fixation mechanism 54”) to fix lead 14 to tissue surrounding lead 14, such as tissue within sacrum 24 in the example of FIG. 1A or tissue at occipital region 29 in the example of FIG. 1B. Fixation mechanism components 54A and 54B are axially displaced from each other along lead body 48. That is, fixation mechanism component 54A has a first axial location along lead body 48, while fixation mechanism component 54B has a second axial location that is different than the first axial location. In the embodiment shown in FIG. 2, the first axial location of fixation mechanism component 54A is a location proximate to electrodes 50, while the second axial location of fixation mechanism component 54B is a location distal to electrodes 50. In particular, fixation mechanism 54 are disposed between electrodes 50 and distal end 48B of lead body 48, between individual electrodes 50A-50D, and between electrodes 50 and proximal end 48A of lead body 48 in order to fix the electrodes in place relative to a target stimulation site.

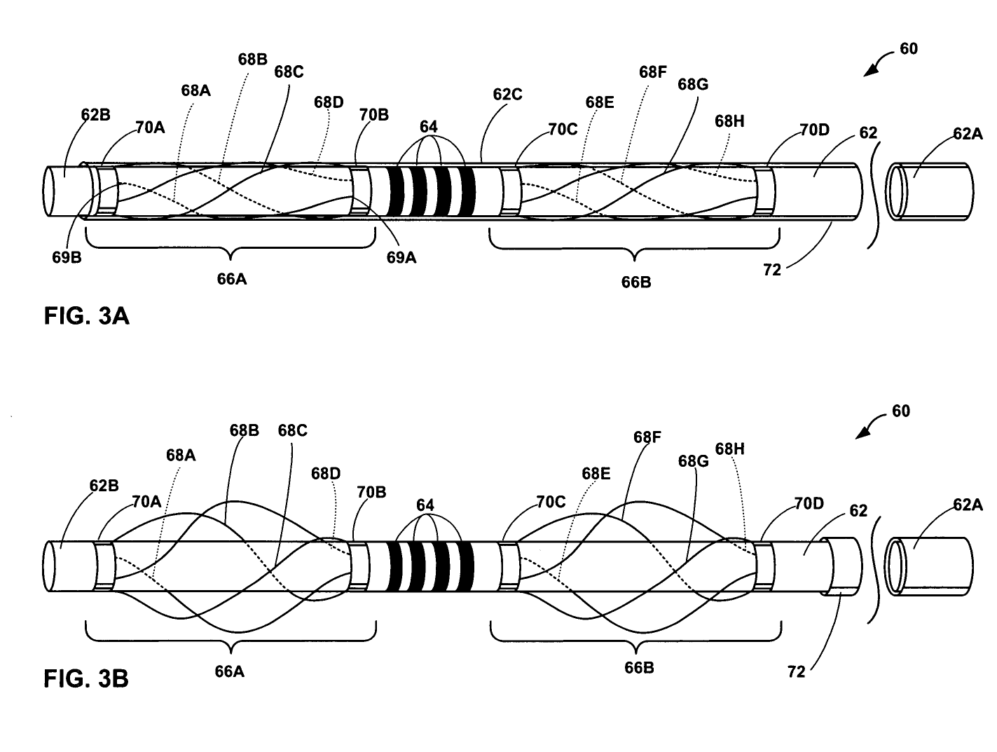

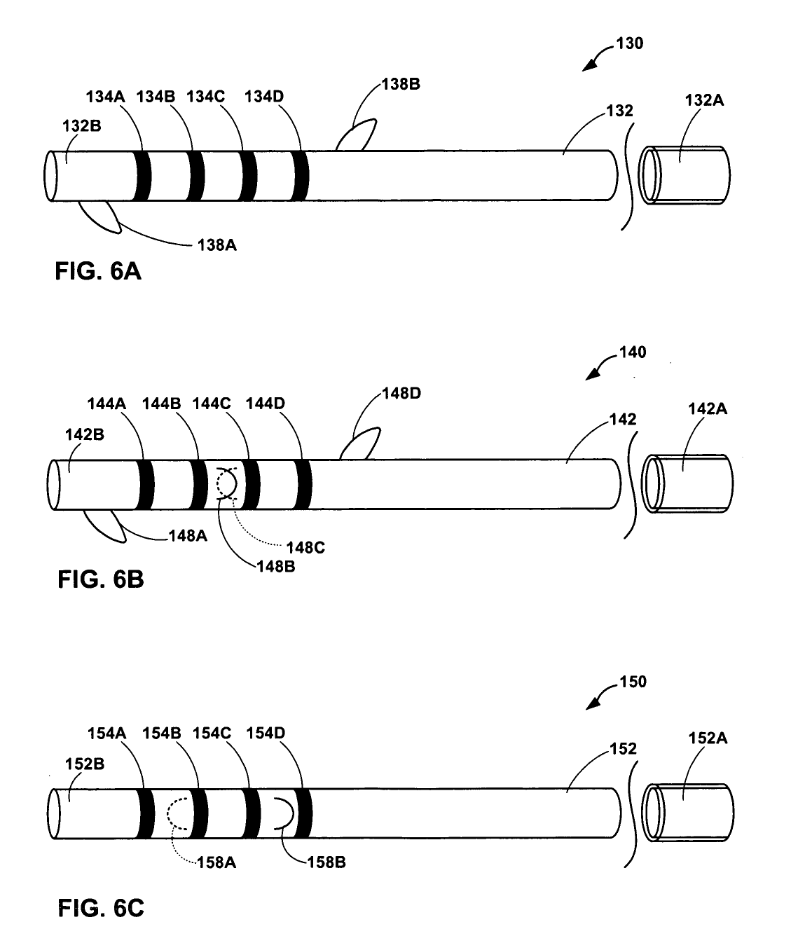

While fixing lead 14 at either the proximal side of the electrodes (e.g., as shown in FIG. 3 of commonly assigned U.S. Patent Application Publication No. 2005/0096718 entitled, “IMPLANTABLE STIMULATION LEAD WITH FIXATION MECHANISM,” which is hereby incorporated by reference in its entirety) or the distal side of the electrodes 50 may be useful in some applications, in other applications, it may be desirable to fix lead 14 at both the proximal and distal sides of electrodes 50, as depicted in FIG. 2. In some applications of therapy system 10, fixing lead 14 on both the proximal and distal side of electrodes 50 may be more desirable than simply fixating lead 14 at one portion of lead body 48. In certain uses of lead 14, fixation mechanism components 54A and 54B located distally and proximally, respectively, to electrodes 50 may provide a more secure attachment than simply fixing lead 14 at one portion of the lead body. By fixing lead 14 on both the proximal and distal sides of the electrodes 50, the portion of lead body 14 containing electrodes 50 may remain more stationary. This may be useful, for example, in an application in which the lead (e.g., lead 130 of FIG. 6A) is a part of a therapy system delivering electrical stimulation to a pudendal nerve of a patient. Alternatively, fixing lead 14 at either one or both of the proximal and distal side of electrodes 50 and between two electrodes, e.g., electrodes 50B and 50C, may more locally fix one or more electrodes to the surrounding tissue.

In accordance with an embodiment of the invention, fixation mechanism 54 may include a plurality of expandable wire-like elements, which may be configured in a substantial helical shape or other shapes. The material of the wire-like elements may have elastic or super-elastic properties. In one embodiment, the material of the wire-like elements may be a shape memory material, such as a shape memory alloy (e.g., nickel-titanium alloys, such as Nitinol) or shape memory polymer.

In one embodiment, for sacral applications, fixation mechanism 54 may be approximately sized to be expandable to a diameter sufficient to fix lead 14 within tissue site posterior to foramen 22. Alternatively, fixation mechanism 54 may facilitate fixation of lead 14 within other tissues target sites, including the epidural region proximate the spine. In those cases, fixation mechanism 54 may be sized to expand to any of a variety of diameters appropriate for engagement of tissue within the desired target site.

In comparison to some existing methods of fixing implanted medical leads, such as suturing lead 14 to surrounding tissue, the wire-like fixation elements of fixation mechanism 54 may permit implantation of lead 14 in patient 16 via a minimally invasive surgery, which may allow for reduced pain and discomfort for patient 16 relative to surgery, as well as a quicker recovery time.

FIG. 3A is a perspective drawing illustrating an exemplary neurostimulation lead 60, which includes lead body 62 extending between proximal end 62A and distal end 62B, a plurality of stimulation electrodes 64, and fixation mechanism components 66A and 66B (collectively “fixation mechanism 66”), which are axially displaced from each other along lead body 62. Proximal end 62A of lead body 62 includes contacts (not shown in FIGS. 3A and 3B) to electrically connect lead 60, and in particular, electrodes 50, to a lead extension or an electrical stimulator (e.g., neurostimulator 12 in FIG. 1A). Lead body 62 and electrodes 64 are similar to lead body 48 and electrodes 50 of FIG. 2. Fixation mechanism 66 includes a plurality of expandable wire-like elements 68A-68H (collectively “wire-like elements 68”) that are configured to expand radially outward from lead body 62 in order to engage with surrounding tissue to help prevent migration of lead 60 from the target stimulation site. While “radially outward” is referred to throughout the disclosure, it should be understood that the expansion of wire-like elements 68 includes both axial and radial components because wire-like elements 68 may extend from lead body 62 at an acute angle with respect to surface 62C of lead body 62. In FIG. 3A, phantom lines are used to indicate the parts of wire-like elements 68 that are behind lead body 62.

In one embodiment, wire-like elements 68 may be configured in a substantial helical shape. As shown in FIG. 3A, fixation mechanism 66 includes eight wire-like elements 68, each having a substantial helical shape. Four wire-like elements 68A-68D are located at a first axial position between the distal end 62B of lead body 62 and electrodes 64, and four additional wire-like elements 68E-68H are located at a second axial position between electrodes 64 and the proximal end 62A of lead body 62. As previously discussed, in some applications of lead 14, providing wire-like fixation elements 68 on both the proximal and distal sides of electrodes 64 may more desirable than simply providing wire-like fixation elements on one portion of lead body 62. For example, by fixing lead 60 on both the proximal and distal sides of electrodes 64, the portion of lead body 62 containing electrodes 64 may remain more stationary.

In some embodiments, wire-like elements 68 may be composed at least in part of a material with elastic or super-elastic properties. In other embodiments, wire-like elements 68 may be composed at least in part of a shape memory alloy, such as Nitinol.

Each wire-like element 68 includes a proximal end and a distal end. For example, wire-like element 68A extends between proximal end 69A and distal end 69B. Proximal ends and distal ends of wire-like elements 68 may be mechanically coupled to lead body 62 by a variety of techniques. In one embodiment, retainer rings 70A, 70B, 70C and 70D (collectively “retainer rings 70”) may be mounted about lead body 62 to retain proximal and distal ends of wire-like elements 68. More specifically, retainer ring 70A retains distal ends of wire-like elements 68A-D, retainer ring 70B retains proximal ends of wire-like elements 68A-D, retainer ring 70C retains distal ends of wire-like elements 68E-H, and retainer ring 70D retains proximal ends of wire-like elements 68E-H. Lead body 62 and retainer rings 70 may include polyurethane or silicone in some embodiments. Alternatively, retainer rings 70 may be formed from a metal. In other embodiments, adhesive bonding, crimping, welding, and the like may be used to secure wire-like elements 68 to lead body 62 in addition to or instead of retainer rings 70. In some embodiments, wire-like elements 68 may be formed integrally with lead body 62.

The points where each of wire-like elements 68 are secured to lead body 62 may be referred to as proximal joints and distal joints. For example, proximal end 69A and distal end 69B of wire-like element 68A may also be referred to as proximal joint 69A and distal joint 69B. Although proximal end 69A and distal end 69B of wire-like element 68A are described in further detail below, the description of wire-like element 68A is also applicable to each of the other wire-like elements 68B-H. In one embodiment, distal joint 69B of wire-like elements 68A may be weaker than proximal joint 69A. This feature, which will be described in more detail below, may be useful when withdrawing neurostimulation lead 60 during explant from a patient. In particular, weakened distal joint 69B may facilitate withdrawal even when there is significant fibrous ingrowth near neurostimulation lead 60 by promoting breakage of fixation mechanism 66.

In practice, fixation mechanism 66 facilitates fixation of neurostimulation lead 60 to surrounding tissue, e.g., within or posterior to sacral foramen 22 (FIG. 1A). Fixation mechanism 66 may be sized to be expandable to a diameter sufficient to fixate lead 60 proximate to a target stimulation site. For example, fixation mechanism may be expandable to a diameter in a range of approximately 2 millimeters (mm) to 10 mm, and in one embodiment, approximately 4 to 6 mm, when disposed within a tissue site proximate sacral foramen 22 in the presence of compressive forces generated by typical tissue. In another embodiment, fixation mechanism 66 may facilitate fixation of neurostimulation lead 60 to tissue surrounding neurostimulation lead 60 in other target sites. If lead 60 is implanted in the epidural region around the spine, for example, fixation mechanism 66 may be expandable to a diameter in a range of approximately 6 mm to 15 mm, and in one embodiment, approximately 9 mm to 12 mm. Also, if fixation mechanism 66 is spring-biased, it may have a different spring force depending on the known tissue characteristics of the intended target site for implantation, e.g., tissue presented by sacral, spinal cord, gastric, deep brain, occipital or other stimulation sites. As an example, the epidural region may present less resistance to expansion than more dense tissue area in other areas.

As described above, neurostimulation lead 60 carries a number of stimulation electrodes 64 to permit delivery of electrical stimulation to a target stimulation site such as a sacral nerve (FIG. 1A) or an occipital nerve (FIG. 1B). In one embodiment, stimulation electrodes 64 may each include at least one electrode. Accordingly, lead body 62 of neurostimulation lead 60 includes one or more conductors to electrically couple electrodes 64 to terminals within neurostimulator 12 (FIG. 1A). In one embodiment, at least one of the wire-like elements 68 may be formed at least in part of an electrically conductive material, thereby enabling the wire-like element to act as an electrode for neurostimulator 12, either as an anode or cathode.

Fixation mechanism 66 is shown in a restrained state in FIG. 3A. In particular, restraint mechanism 72 is shown restraining expandable fixation mechanism 66 against expansion. Restraint mechanism 72 shown in FIG. 3A includes a lead introducer, which defines an inner lumen that is sized to accommodate stimulation lead body 62 and fixation mechanism 66. Alternatively, restraint mechanism 72 may be a sheath. When fixation mechanism 66 is within restraint mechanism 72, restraint mechanism 72 encloses fixation mechanism 66 and forces fixation mechanism 66 into a compressed state. Restraining fixation mechanism 66 permits restraint mechanism 72 and stimulation lead 60 to retain a small overall lead diameter during lead implantation, which may minimize the invasiveness of a procedure for implanting lead 60 in a patient because a small diameter introducer needle may be used to implant the small overall lead 60 diameter. In this manner, fixation mechanism 66 may be restrained from expansion and may be deployed via an introducer needle or other minimally invasive delivery device. Introducing fixation mechanism 66 via a needle requires only minimally invasive techniques, which may allow for a shorter recovery for the patient.

In one embodiment, at least a portion of neurostimulation lead 60, such as a portion of lead body 62, may include radio-opaque material that is detectable by imaging techniques, such as fluoroscopic imaging or x-ray imaging. This feature may be helpful for maneuvering neurostimulation lead 60 relative to a target site within the body. For example, the distal end of neurostimulation lead 60 may include radio-opaque material that is visible via fluoroscopic imaging. Radio-opaque markers, as well as other types of markers, such as other types of radiographic and/or visible markers, may also be employed to assist a clinician during the introduction and withdrawal of neurostimulation lead 60 from a patient.

FIG. 3B is a perspective drawing illustrating an exemplary neurostimulation lead 60 with fixation mechanism 66 in an expanded state, in which wire-like elements 68 extend from lead body 62 to enable fixation mechanism 66 to engage with surrounding tissue to substantially fix neurostimulation lead 60 proximate to a target stimulation site. Restraint mechanism 72 is shown partially withdrawn from lead body 62. Upon withdrawal of restraint mechanism 72, fixation mechanism 66 are exposed and wire-like elements 68 expand radially outward from the lead body 62. Wire-like elements 68 expand outward in response to spring force provided by the elastic, superelastic, or shape memory properties of elements 68. Fixation mechanism 66 may be expandable to any suitable diameter, which may depend on the particular stimulation application of lead 60. In one embodiment, the diameter of fixation mechanism 66 may be expandable to approximately 2 mm to 10 mm. For example, the diameter of fixation mechanism 66 may be expandable to approximately 4 to 6 mm. In another embodiment, the diameter of fixation mechanism 66 may be expandable to a larger diameter, e.g., for epidural implantation. The larger diameter may be approximately 6 mm to 15 mm, and in one embodiment, approximately 9 mm to 12 mm, as discussed above.

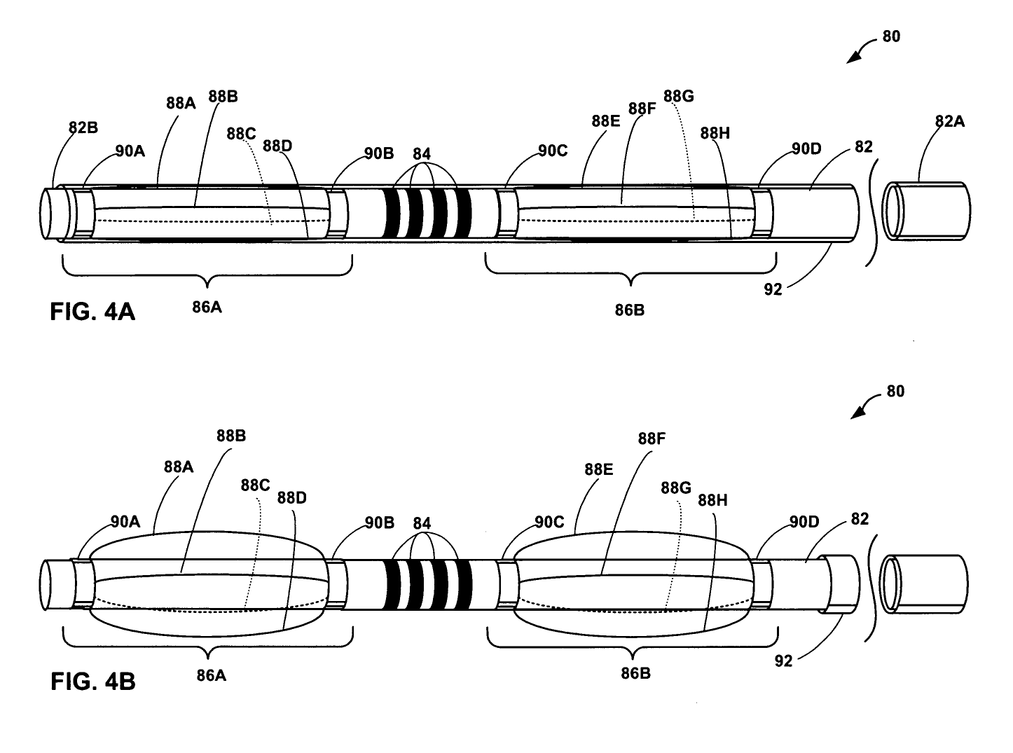

FIG. 4A is a perspective drawing illustrating an alternate neurostimulation lead 80 with alternative fixation mechanism components 86A and 86B (collectively “fixation mechanism 86”) attached to lead body 82. Lead body 82 is similar to lead body 62 of FIGS. 3A and 3B, and extends between proximal end 82A and distal end 82B. Electrodes 84, which are similar to electrodes 64 of FIGS. 3A and 3B, are disposed near distal end 82B of lead body 82. Additionally, like proximal end 62A of lead 60, proximal end 82A of lead body 82 includes contacts (not shown in FIGS. 4A and 4B) that are used to electrically connect electrodes 84 of lead 80 to a lead extension or an electrical stimulator (e.g., neurostimulator 12 in FIG. 1A).

Fixation mechanism 86 includes wire-like elements 88A-88H (collectively “wire-like elements 88”), which are restrained by restraint mechanism 92. As described above with respect to restraint mechanism 72 of FIGS. 3A and 3B, restraint mechanism 92 may be any suitable apparatus for restraining expansion of wire-like elements 88, such as, but not limited to a lead introducer or a sheath. In FIG. 4A, phantom lines are used to indicate the parts of wire-like elements 88 that are behind lead body 82. Again, neurostimulation lead 80 is very similar to neurostimulation lead 60, with the main difference being the configuration of wire-like elements 88.

Fixation mechanism 86 of FIG. 4A is similar to fixation mechanism 66 of FIGS. 3A and 3B, but has a different shape. In particular, wire-like elements 88 do not cross each other as wire-like elements 68 of FIG. 3A each having a helical configuration cross each other. Instead, FIG. 4A shows eight wire-like elements 88 with ends that may be, but need not be, evenly spaced around the periphery of lead body 82. Four wire-like elements 88A-88D are located at a first axial position between distal end 82B of lead body 82 and electrodes 84, and four additional wire-like elements 88E-88H are located at a second axial position between electrodes 84 and proximal end 82A of lead body 82. Providing wire-like fixation elements 88 on both the proximal and distal sides of electrodes 84 may be more desirable than simply fixing lead 80 at one portion of lead body 82. In certain uses of lead 80, fixation mechanism components 86A and 86B located distally and proximally, respectively, to electrodes 84 may provide a more secure attachment than simply fixing lead 80 at one portion of lead body 82. By fixing lead 80 on both the proximal and distal sides of the electrodes 84, the portion of lead body 80 containing electrodes 84 may remain more stationary. This may be useful, for example, in an application in which the lead (e.g., lead 130 of FIG. 6A) is a part of a therapy system delivering electrical stimulation to a pudendal nerve of a patient.

FIG. 4B is a perspective drawing illustrating an exemplary neurostimulation lead 80 with fixation mechanism 86 in an expanded state, which enables fixation mechanism 86 to engage with surrounding tissue in order to fix a position of an implanted neurostimulation lead 80 proximate to a target stimulation site. Restraint mechanism 92 is shown partially withdrawn from lead body 82. Withdrawing restraint mechanism 92 allows wire-like elements 88 to expand. As with fixation mechanism 66 of FIGS. 3A-3B, fixation mechanism 86 of FIGS. 4A and 4B may expand to any suitable dimension.

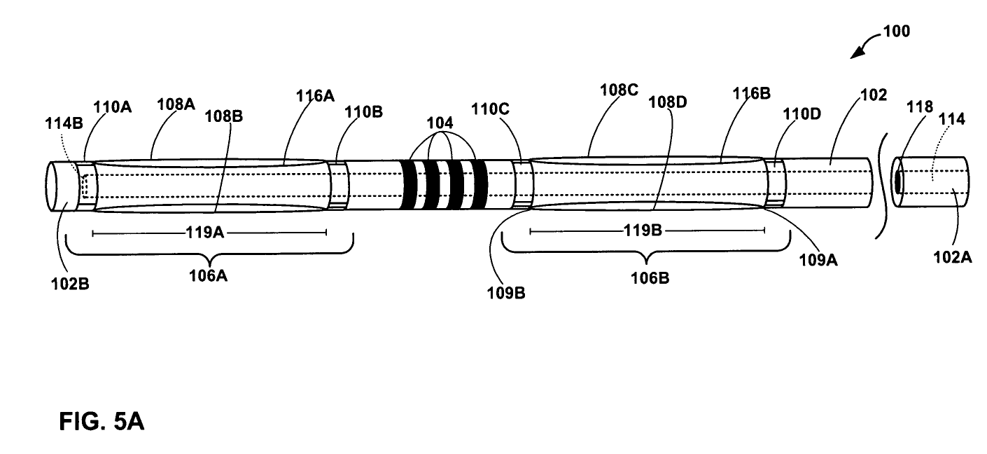

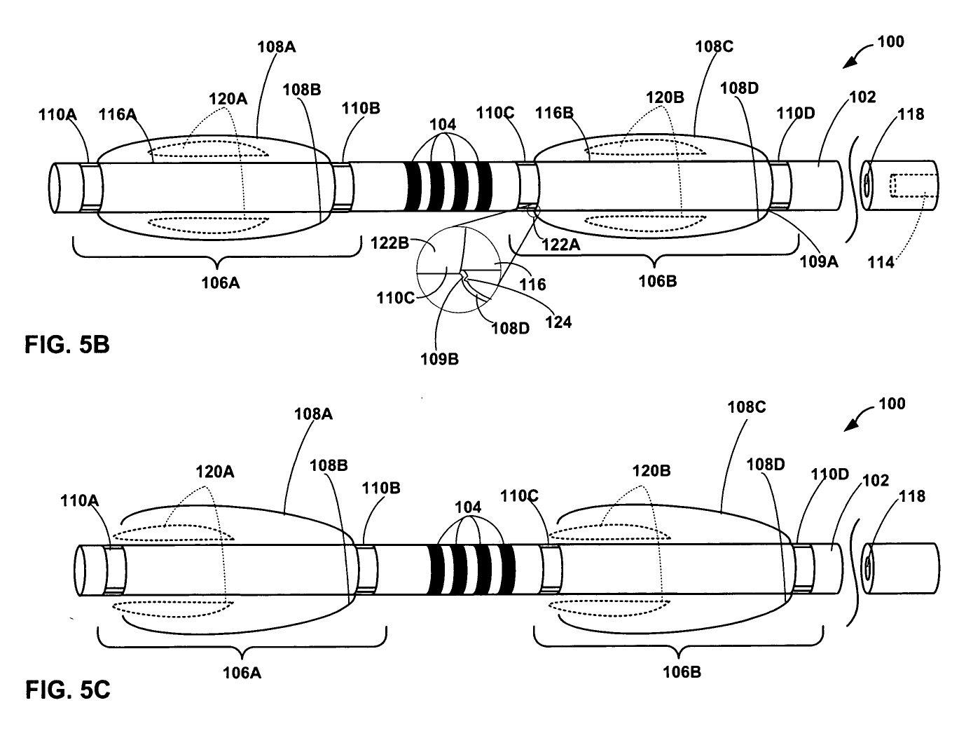

FIG. 5A is a perspective drawing illustrating another embodiment of neurostimulation lead 100, which includes lead body 102, one or more stimulation electrodes 104, and fixation mechanism components 106A and 106B (collectively “fixation mechanism 106”), which include a number of expandable wire-like elements 108A-108D (collectively “wire-like elements 108”). In FIG. 5A, phantom lines are used to indicate the parts of wire-like elements 108 that are behind lead body 102. As described above, fixation mechanism 106 may be mounted to lead 100 to fixate lead 100 to tissue surrounding lead 100, such as tissue posterior to foramen 22 of sacrum 24 (FIG. 1A). Additionally, proximal end 102A of lead body 102 includes contacts (not shown in FIGS. 5A-5C) that are used to electrically connect electrodes 104 of lead 100 to a lead extension or an electrical stimulator (e.g., neurostimulator 12 in FIG. 1A).

Wire-like elements 108 of neurostimulation lead 100 may come in many configurations. As shown in FIG. 5A, neurostimulation lead 100 may include four uncrossed wire-like elements 108 with proximal and distal ends that may be, but need not be, evenly spaced around the periphery of lead body 102. In other embodiments, one or more of wire-like elements 108 of expansion mechanism 106 may be configured in a substantial helical shape (e.g., as shown with respect to each of wire-like elements 68 of FIGS. 3A and 3B). In addition, fixation mechanism 106 may include retainer rings 110A, 110B, 110C, and 110D (collectively “retainer rings 110”), which are similar to retainer rings 70 of FIGS. 3A and 3B. In particular, retainer rings 110A and 110B may secure distal ends and proximal ends, respectively, of wire-like elements 108A and 108B to lead body 102, while retainer rings 110C and 110D may secure distal ends and proximal ends, respectively, of wire-like elements 108C and 108D to lead body 102.

Lead body 102 of neurostimulation lead 100 is shown with an inner lumen 118 that accommodates a restraint mechanism, such as stylet 114. A distal end 114B of stylet 114 bears against a surface within lead body 102 to exert a linear force along the length of the lead body 102 (where the length is generally measured in a direction along lead body 102 proximal end 102A to distal end 102B of lead body 102) to cause lead body 102 to straighten out. In some embodiments, lead body 102 may include one or more portions 116A and 116B (collectively “portions 116”) that are formed from an elastic material, causing the diameter of portions 116 to decrease when portions 116 of lead body 102 are stretched. Elastic portions 116 of the lead body 102 are shown in FIG. 5A in a restrained state, where the diameters of the stretched elastic portions 116 are smaller than the remainder of lead body 102 that is not stretched.

Stretching lead body 102 allows wire-like elements 108 to lengthen and straighten out, as shown in FIG. 5A. In other words, wire-like elements 108 of fixation mechanism 106 may be restrained from expansion by straightening lead body 102. However, wire-like elements 108 of fixation mechanism 106 may be also restrained from expansion via a restraint mechanism such as an introducer needle or sheath (e.g., restraint mechanism 72 of FIGS. 3A and 3B). Regardless of the restraint mechanism, lead 100 may be implanted into a patient via an introducer needle or other minimally invasive techniques.

In some embodiments, elastic portions 116 of lead body 102 may be provided and stretched under axial force from stylet 114, thereby increasing the linear distance 119A between the proximal and distal ends of wire-like elements 108A and 108B as well as the linear distance 119B between the proximal and distal ends of wire-like elements 108C and 108D. For example, when elastic portion 116A is stretched, the linear distance between proximal end 109A and distal end 109B of wire-like element 108D is linear distance 119B. Relaxing elastic portions 116 of lead body 102, e.g., by retracting the stylet 114, causes lead body 102 to decrease in length, permitting wire-like elements 108 to extend radially outward from lead body 102, as shown in FIG. 5B.

FIG. 5B is a perspective drawing illustrating neurostimulation lead 100 of FIG. 5A with fixation mechanism 106 in an expanded state, which enables wire-like elements 108 of fixation mechanism 106 to engage with surrounding tissue in order to fix a position of an implanted neurostimulation lead 100 proximate to a target stimulation site. As FIG. 5B illustrates, restraining fixation mechanism 106 by extension of stylet 114 allows for relatively large stimulation zones while still retaining a small overall lead diameter during lead deployment because of the expansion capacity of wire-like elements 108 of fixation mechanism 106 once stylet 114 is withdrawn from lead body 102. In FIG. 5B, stylet 114 is shown partially withdrawn from lead body 102. Withdrawing stylet 114 from inner lumen 118 of lead 100 allows wire-like elements 108 to expand. In particular, stylet 114 may initially extend lead body 102 substantially straight so that wire-like elements 108 are also pulled straight and are restrained against expansion. In some embodiments, stylet 114 may exert axial force along the longitudinal axis of lead body 102 to thereby stretch at least a portion of lead body 102. Upon withdrawal of stylet 114, the spring force exerted by wire-like elements 108 causes the wire-like elements to expand radially outward. Again, the diameter of fixation mechanism 106 may be expandable to a range of diameters appropriate for different target sites, as described above.

After neurostimulation lead 100 has been implanted within a patient for a considerable amount of time, fibrous ingrowths 120A and 120B (collectively “fibrous ingrowths 120) may develop around neurostimulation lead 100. For example, as shown in FIG. 5B, fibrous ingrowths 120A and 120B may develop between lead body 102 and wire-like elements 108A-108D of fixation mechanism 106. Due to the fibrous ingrowths 120, resistance may be encountered if withdrawal of neurostimulation lead 100 from the patient is attempted. An embodiment of the invention may provide a feature to reduce resistance and to limit further problems due to the fibrous ingrowths 120.

As described above, the points where wire-like elements 108 are secured to lead body 102 may be referred to as proximal joints and distal joints. For example, proximal end 109A and distal end 109B of wire-like element 108D may also be referred to as proximal joint 109A and distal joint 109B, respectively. In one embodiment, the distal joint of each wire-like element 108 may be intentionally made weaker than the proximal joint. Circle 122B provides an enlarged representation of circle 122A. As shown in the enlarged view 122B, the distal joint 109B of wire-like element 108D, which is adjacent to retainer ring 110C, may be intentionally thinned to create a breakpoint 124 that causes wire-like element 108D to break under sufficient force. For example, distal joint 109B may be engineered to be weaker than the proximal joint by perforating, scoring, thinning, or otherwise working the distal joint to break away under force generated by withdrawal of lead 100 from a target stimulation site. This feature may be useful when withdrawing neurostimulation lead 100 from fibrous ingrowths 120. In practice, the relatively weak distal joints of wire-like elements 108 may disconnect from lead body 102, while the relatively strong proximal joints of wire-like elements 108 may remain connected to lead body 102.

Any suitable technique for achieving weakened distal joints of each of wire-like elements 108 may be used. For example, in embodiments in which each of wire-like elements 108 are adhered to lead body 102, a stronger adhesive may be used to couple the proximal end of each wire-like element 108 to lead body 102, such that the distal end of the wire-like elements 108 are inclined to break away from lead body 102 before the proximal end. Or, in another embodiment, distal end retainer rings 110A and 110C may be formed to release the distal end of each of wire-like elements 108 under sufficient pulling force (which may be exerted on the distal end of each wire-like element 108 during withdrawal of lead 100 from a patient).

As FIG. 5C illustrates, after the distal joints of wire-like elements 108 are disconnected from lead body 102, neurostimulation lead 100 may be withdrawn from the patient, leaving fibrous ingrowths 120 behind. If there are no substantial fibrous ingrowths 120, it may be possible to withdraw neurostimulation lead by simply restraining fixation mechanism 106 (as in FIG. 5A), i.e., returning the fixation mechanism from its expanded configuration to it restrained configuration, which may serve to loosen neurostimulation lead 100 from its fixated state.

The leads depicted in FIGS. 3A, 3B, 4A, 4B, 5A, 5B and 5C, are illustrative of example embodiments of the present invention. The number, configuration, and location of the wire-like elements of the fixation mechanism are not limited to the embodiments shown is FIGS. 3A, 3B, 4A, 4B, 5A, 5B and 5C. For example, the wire-like elements may be configured in a variety of other designs or placed at any location along the lead body.

FIGS. 6A-6C are perspective views of leads including alternate configurations of fixation mechanisms for substantially fixing positions of the respective leads in accordance with the invention. The leads illustrated in FIGS. 6A-6C are shown in their expanded states but are capable of being restrained with a restraining device, such as an introducer lumen or stylet as previously described. Additionally, the proximal end of each lead body includes contacts (not shown in FIGS. 6A-6C) that are used to connect electrodes of each lead to a lead extension or an electrical stimulator (e.g., neurostimulator 12 in FIG. 1A).

FIG. 6A illustrates lead 130, which includes lead body 132 extending between proximal end 132A and distal end 132B and electrodes 134A-134D disposed proximate to distal end 132B of lead body 132. As FIG. 6A illustrates, in one embodiment of the invention, there may be as few as two wire-like elements 138A and 138B mounted on lead body 132 to form a fixation mechanism. As shown in FIG. 6A, one wire-like element 138A may be located at a first axial position with respect to lead body 132 and a second wire-like element 138B may be located at a second axial position with respect to lead body 132. Wire-like element 138A is located on a portion of lead body 132 proximal to electrodes 134A-134D and at least one wire-like element is located on a portion of lead body 132 distal to electrodes 134A-134D. More specifically, wire-like element 138A is disposed between the most distally located electrode 134A and distal end 132B of lead body 132, and wire-like element 138B is disposed between the most proximally located electrode 134D and proximal end 132A of lead body 132. Alternatively, one or more of the wire-like elements may be disposed in between individual electrodes 134, e.g., between electrodes 134A and 134B.

In one embodiment, wire-like elements 138A and 138B may extend from only one side of the lead body, rather than being distributed about the periphery of lead body 132. FIG. 6A further illustrates an embodiment of lead 130 fixation mechanism in which wire-like elements 138A and 138B located at different axial positions with respect to lead body 132 extend from different sides of lead body 132. More specifically, FIG. 6A illustrates first wire-like element 138A located at a first axial position extending in a first direction, and second wire-like element 138B located at a second axial position extending in a second direction that differs from the first direction. In FIG. 6A, wire-like elements 138A and 138B extend in approximately opposite directions. However, in other embodiments, wire-like elements 138A and 138B may each extend in directions that are not approximately opposite each other.

FIG. 6B illustrates another embodiment of lead 140, which includes lead body 142 extending between proximal end 142A and distal end 142B, and electrodes 144A-144D disposed proximate to distal end 142B of lead body 142. Lead 140 includes wire-like element 148A located between distal end 142B of lead body 142 and electrodes 144A-144D (i.e., on the “distal side” of electrodes 144A-144D), wire-like elements 148B and 148C (148B shown with phantom lines in FIG. 6B) located between electrodes 144B and 144C, and wire-like element 148C located between the proximal end 142A of lead body 142 and electrodes 144A-144D (i.e., on the “proximal side” of electrodes 144A-144D). Fixating the lead between two electrodes 144B and 144C may more locally fix one or more of the electrodes to the surrounding tissue.

As an additional alternative, a lead may only include wire-like elements between electrodes to ensure fixation of the one or more electrodes proximate to a target stimulation site, as shown in FIG. 6C. FIG. 6C is a perspective view of lead 150, which includes lead body 152, electrodes 152A-152D, wire-like element 158A (shown in FIG. 6C with phantom lines) located between electrodes 154A and 154B, and wire-like element 158B located between electrodes 154C and 154D. Wire-like elements 158A and 158B are generally disposed on opposite sides of lead body 152. This configuration may locally fixate electrodes 154B and 154C as well as generally fixate lead 150. Locally fixating electrodes 154B and 154C may be useful in applications where a clinician aims to implant lead 150 such that the mid-length of electrode 154A-154D region of lead body 152, i.e., the location between electrodes 154B and 154C, is centered at the target stimulation site. Alternatively, wire-like elements 158A and 158B may be positioned to locally secure various electrodes (e.g., between electrodes 154B and 154C).

In general, a plurality of wire-like elements may be used in fixating a lead, and at least one wire-like element may be separated from at least one other wire-like element by at least one electrode. Additionally, other forms of fixation elements may be used in addition to balloons. The additional fixation elements may be any suitable actively or passively deployed fixation element that helps prevent migration of lead 100 when lead 100 is implanted in patient 16, such as, but not limited to, one or more tines, barbs, hooks, wire-like elements, adhesives (e.g., surgical adhesives), balloon-like fixation elements, pinning fixation elements, collapsible or expandable fixation structures, and so forth. The fixation elements may be composed of any suitable biocompatible material, including, but not limited to, polymers, titanium, stainless steel, Nitinol, other shape memory materials, hydrogel or combinations thereof. Examples of suitable tines include, but are not limited to, the tines described in commonly-assigned U.S. Pat. No. 6,999,819, entitled, “IMPLANTABLE MEDICAL ELECTRICAL STIMULATION LEAD FIXATION METHOD AND APPARATUS,” which issued on Feb. 14, 2006 and is hereby incorporated by reference in its entirety. If additional fixation elements are used in addition to wire-like elements, all of the fixation elements may be restrained using a restraint element during implantation of the lead and expanded upon implantation. Also, all of the fixation mechanisms may be configured to permit explant.

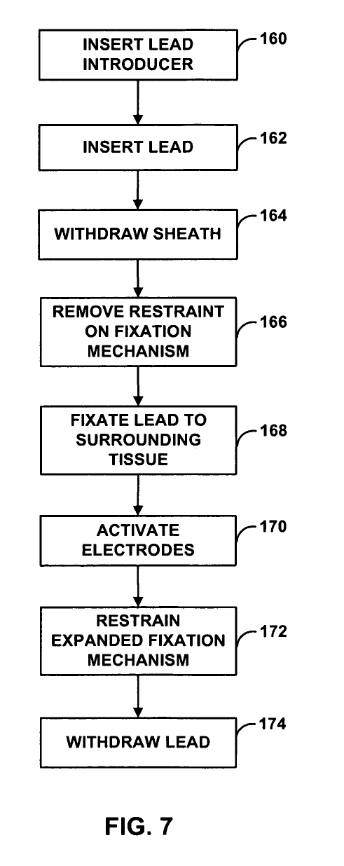

FIG. 7 is a flow diagram illustrating a process for percutaneously implanting a lead including a fixation mechanism in accordance with one embodiment of the invention. While the process shown in FIG. 7 is described with respect to lead 60 of FIGS. 3A and 3B, in other embodiments, the lead may be, for example, any one of leads 14, 80, 100, 130, 140 or 150 of FIGS. 2, 4A, 5A, and 6A-6C, respectively. In addition, the process shown in FIG. 7 may be used to implant any suitable lead including a fixation mechanism with expandable wire-like elements in accordance with the invention.

Initially, an introducer needle assembly is inserted into a patient (160). The needle assembly may include a needle and an introducer stylet fitted into a lumen defined by the needle. In one embodiment, the lumen has a diameter between 14 and 20 gauge to allow the needle to receive the introducer stylet. The introducer stylet may fill the lumen of the needle, preventing tissue coring. In some instances, the needle may include a straight needle for sacral implantation or a modified Tuohy needle for epidural applications, which has an opening that is angled approximately 45 degrees so that an instrument passing through the needle exits at an angle.

The neurostimulation lead introducer may be inserted (160) by a variety of techniques not limited to the technique described above. Lead 60 is inserted (162) and advanced through the lead introducer. Lead 60 is typically advanced through the introducer until electrodes 50 reach tissue proximate to the target stimulation site. Meanwhile, a restraint mechanism, such as the lead introducer, a sheath other than the lead introducer, a stylet, or the like, restrains expansion of expandable fixation mechanism 66 to prevent premature radial expansion of wire-like elements 68.

In one embodiment, the restraint mechanism for fixation mechanism 66 includes the lead introducer. In this case, the act of withdrawing the lead introducer removes the restraint on fixation mechanism 66 (166). In another embodiment, the restraint mechanism includes a stylet (e.g., stylet 114 of FIGS. 5A-5C) that may extend through a lumen of neurostimulation lead 66, causing part of lead body 62 to straighten, lengthen or stretch, and allowing the wire-like elements 66 of fixation mechanism 66 to be restrained against lead body 62 of neurostimulation lead 60. In this case, removing the stylet, which acts as a restraint mechanism, removes the restraint on fixation mechanism 66, thereby enabling wire-like elements 68 to expand (166).

Thus, after lead 60 has been properly placed proximate to a target stimulation site, the restraint mechanism is removed from fixation mechanism 66, allowing wire-like elements 68 to expand. Upon expansion, wire-like elements 68 engage with surrounding tissue, thereby fixing neurostimulation lead 60 proximate to the target stimulation site (168). Fixating neurostimulation lead 60 to surrounding tissue may prevent detrimental effects that may result from a migrating neurostimulation lead 60.

After lead 60 is fixed proximate to the target stimulation site, electrodes 64 on the neurostimulation lead 60 may be activated (170) to provide therapy to the patient, e.g., by coupling proximal end 62A of neurostimulation lead body 62 to a neurostimulator (e.g., neurostimulator 12 of FIGS. 1 and 2). In one embodiment, a lead extension may be provided to couple the neurostimulation lead to the neurostimulator.

Therapy may require that neurostimulation lead 60 be activated for only a short period of time, e.g., for trial stimulation, sometimes referred to as screening. On the other hand, therapy may require that neurostimulation lead 60 be implanted chronically for a number of years. In either case, it may become necessary to remove neurostimulation lead 60 from the patient. The expanded fixation mechanism 66 may be restrained as it was when it was inserted (172), and neurostimulation lead 60 may be withdrawn from the patient (174). As described above, it may be helpful to disconnect the distal joints of wire-like elements 68. For example, wire-like element 68A may be disconnected from lead body 62 at distal joint 69B, leaving proximal joint 69A intact. This feature may be useful when withdrawing neurostimulation lead 60 from fibrous ingrowth. In practice, the relatively weak distal joints of wire-like elements 68 may disconnect from lead body 62, while the relatively strong proximal joints of wire-like elements 68 may remain connected to lead body 62. With distal joints of wire-like elements 68 disconnected, neurostimulation lead 60 may be withdrawn from the patient, leaving the fibrous ingrowth behind.

A lead including wire-like fixation elements may be useful for various electrical stimulation systems. For example, the lead may be used to deliver electrical stimulation therapy to patients to treat a variety of symptoms or conditions such as chronic pain, tremor, Parkinson's disease, multiple sclerosis, spinal cord injury, cerebral palsy, amyotrophic lateral sclerosis, dystonia, torticollis, epilepsy, pelvic floor disorders, gastroparesis, muscle stimulation (e.g., functional electrical stimulation (FES) of muscles) or obesity. In addition, the fixation element arrangement described herein may also be useful for fixing a catheter, such as a drug deliver catheter, proximate to a target drug delivery site.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems and leads for neurostimulation, as described herein. Also, the leads described herein may have a variety of neurostimulation applications, as well as possible applications in other electrical stimulation contexts, such as delivery of cardiac electrical stimulation, including paces, pulses, and shocks.

Many embodiments of the invention have been described. Various modifications may be made without departing from the scope of the claims. These and other embodiments are within the scope of the following claims.

1. An apparatus comprising:
an implantable elongated member configured to be coupled to a medical device to deliver a therapy from the medical device to a target therapy delivery site in a patient; and
a fixation mechanism mechanically coupled to the elongated member, the fixation mechanism comprising:
a first wire-like element configured to expand to engage with tissue of the patient; and
a second wire-like element axially displaced from the first wire-like element along a length of the elongate member and configured to expand to engage with the tissue of the patient.

2. The apparatus of claim 1, wherein the elongated member comprises a lead comprising a lead body extending between a proximal end and a distal end, and one or more electrodes proximate to the distal end of the lead body.

3. The apparatus of claim 2, wherein the first wire-like element is separated from the second wire-like element by at least one of the electrodes.

4. The apparatus of claim 2, wherein the first wire-like element is mechanically coupled to the lead body at a first position between the one or more electrodes and the proximal end of the lead body and a second wire-like element is mechanically coupled to the lead body a second position between the one or more electrodes and the distal end of the lead body.

5. The apparatus of claim 1, wherein the medical device comprises at least one of a sensor to sense a parameter of a patient, an electrical stimulator or a fluid delivery device.

6. The apparatus of claim 1, wherein the elongated member comprises a catheter configured to deliver a fluid from the medical device to the target therapy delivery site.

7. The apparatus of claim 1, wherein each of the first and second wire-like elements is formed at least in part of at least one of an elastic material, a super-elastic material or a shape memory material.

8. The apparatus of claim 1, wherein each of the wire-like elements comprises a proximal joint where the proximal end of the wire-like element meets the elongated member, and a distal joint where the distal end of the wire-like element meets the elongated member, wherein the distal joint is weaker than the proximal joint.

9. The apparatus of claim 1, further comprising a restraint mechanism to restrain the wire-like elements against expansion, wherein the wire-like elements expand upon removal of at least part of the restraint mechanism.

10. The apparatus of claim 9, wherein the restraint mechanism includes an introducer defining an introducer lumen sized to accommodate the elongated member.

11. The apparatus of claim 9, wherein the restraint mechanism includes a stylet configured to be received in an inner lumen of the elongated member.

12. The apparatus of claim 1, wherein at least a portion of the elongated member is elastic, causing a diameter of the elongated member portion to decrease when the elongated member portion is stretched.

13. The apparatus of claim 1, wherein at least a part of each of the wire-like elements is configured in a substantial helical shape.

14. The apparatus of claim 1, further comprising retainer rings mounted about the elongated member to retain opposite ends of each of the wire-like elements and mechanically couple each of the wire-like elements to the elongated member.

15. The apparatus of claim 1, wherein the medical device is an electrical stimulator and at least one of the wire-like elements acts as an electrode for delivering a stimulation current from the electrical stimulator to the target therapy delivery site.

16. The apparatus of claim 1, wherein the fixation mechanism is sized to be expandable to a diameter in a range of approximately 2 millimeters to 15 millimeters.

17. The apparatus of claim 1, further comprising a radio-opaque material that is detectable by fluoroscopic imaging located on at least a portion of the elongated member.

18. The apparatus of claim 1, wherein the fixation mechanism further comprises a third wire-like element configured to expand to engage with tissue at the target therapy delivery site.

19. An electrical stimulation system comprising:
an implantable electrical stimulator;
a lead comprising:
a lead body having a proximal end and a distal end;
at least one stimulation electrode located proximate to the distal end of the lead body and electrically coupled to the electrical stimulator, wherein the electrical stimulator delivers electrical stimulation to a target stimulation site via the at least one stimulation electrode; and
a fixation mechanism mechanically coupled to the lead body, the fixation mechanism comprising a first wire-like element and a second wire like element separated from the first wire-like element by at least one stimulation electrode, wherein the first and second wire-like elements are each expandable to substantially fix the lead body at the target stimulation site.

20. The electrical stimulation system of claim 19, wherein the first wire-like element is mounted to the lead body at a position between a most proximally located electrode and the proximal end of the lead body and a second wire-like element is located at a position between a most distally located electrode and the distal end of the lead body.

21. The electrical stimulation system of claim 19, wherein the at least one stimulation electrode comprises at least two electrodes, and wherein at least one of the wire-like elements is located between the at least two electrodes.

22. The electrical stimulation system of claim 19, wherein each of the wire-like elements is formed at least in part of at least one of an elastic material, a super-elastic material or a shape memory material.

23. The electrical stimulation system of claim 19, each of the wire-like elements having a proximal joint where the proximal end of the wire-like element meets the lead body, and a distal joint where the distal end of the wire-like element meets the lead body, wherein the distal joint is weaker than the proximal joint.

24. The electrical stimulation system of claim 19, further comprising a restraint mechanism to restrain the wire-like elements against expansion, wherein the wire-like elements expand upon removal of at least part of the restraint mechanism.

25. The electrical stimulation system of claim 19, wherein at least a portion of the lead body is elastic, causing a diameter of the lead body portion to decrease when the lead body portion is stretched.

26. The electrical stimulation system of claim 19, further comprising retainer rings mounted about the lead body to mechanically couple opposite ends of each of the wire-like elements to the lead body.

27. A method comprising:
inserting an elongated member into a patient, wherein the elongated member includes a fixation mechanism mechanically coupled to the elongated member, the fixation mechanism comprising:
a first wire-like element configured to expand to engage with tissue to substantially fix the elongated member proximate to a target therapy delivery site; and
a second wire-like element configured to expand to engage with tissue to substantially fix the elongated member proximate to the target therapy delivery site, wherein the first wire-like element is axially displaced from the second wire-like element; and
removing a restraint mechanism on the fixation mechanism, thereby permitting the wire-like elements to expand and extend from the elongated member.

28. The method of claim 27, wherein inserting the elongated member into the patient comprises inserting an introducer into the patient and inserting the elongated member into the introducer.

29. The method of claim 28, wherein inserting the introducer into the patient comprises subcutaneously introducing the introducer proximate to a peripheral nerve of the patient.

30. The method of claim 29, wherein inserting the introducer proximate to the peripheral nerve comprises positioning the introducer substantially transversely across an occipital nerve.

31. The method of claim 27, wherein removing the restraint mechanism includes withdrawing at least part of a stylet from a lumen of the elongated member, thereby releasing the fixation mechanism to expand.

32. The method of claim 27, wherein the restraint mechanism comprises the introducer, the introducer defining a lumen sized to accommodate the elongated member and wherein removing the restraint mechanism includes withdrawing at least a portion of the introducer, thereby releasing the fixation mechanism to expand.

33. The method of claim 27, further comprising:
detaching a distal end of each wire-like element; and
withdrawing the elongated member from the target site.

34. The method of claim 27, further comprising:
restraining the expanded fixation mechanism; and
withdrawing the elongated member from the target site.

35. The method of claim 27, wherein the elongated member comprises at least one of a lead comprising an electrode or a catheter.

36. The method of claim 27, further comprising coupling the elongated member to a medical device, the medical device delivering a therapy to the target therapy delivery site via the elongated member.

37. The method of claim 27, wherein the elongated member comprises a lead comprising a lead body extending between a proximal end and a distal end, and a first electrode and a second electrode disposed on the lead body proximate to the distal end of the lead body, wherein first wire-like element is mounted to the lead body at a position between the first electrode and the proximal end of the lead body and the second wire-like element is located at a position between the second electrode and the distal end of the lead body.
